(19) United States
(12) Patent Application Publication
(10) Pub. No.: US 2015/0335463 A1
(43) Pub. Date: Nov. 26, 2015

(54) PROFILE FOR THE ATTACHMENT OF AN IMMOBILISATION MASK

(71) Applicant: ORFIT INDUSTRIES, Wijnegem (BE)

(72) Inventor: Simon DE GRUYTERE, Knokke (BE)

(73) Assignee: ORFIT INDUSTRIES, WIJNEGEM (BE)

(21) Appl. No.: 14/648,892

(22) PCT Filed: Dec. 10, 2013
(86) PCT No.: PCT/IB2013/060779

§ 371 Date: Jun. 1, 2015

(30) Foreign Application Priority Data
Dec. 10, 2012 (BE) 2012/0832

(51) Int. Cl. A61F 5/37

(52) U.S. Cl. CPC A61F 5/3707; A61F 5/3769

(57) ABSTRACT

This invention concerns an immobilisation mask, wherein the immobilisation mask (2) comprises a sheet of thermoplastic material for receiving the body part to be immobilized, and is provided with connection means (21) for connecting the immobilisation mask (2) with the carrier (3). The connection means comprise at least one profile (1) that is provided on a first side with first connection means (5, 15, 21) for connecting the profile to the carrier (3), wherein the first connection means (5, 15) are provided, in the connected state, to engage corresponding connection means along the outer edge (4) of the carrier (3), and on a second side opposite the first side, second connection means (22) for connecting the profile with the immobilisation mask. The first connection means (5, 15, 21) are provided, in the connected state, to engage corresponding connection means (13) along the outer edge (4) of the carrier (3).

This invention concerns an assembly of a carrier for supporting a patient and an immobilisation mask that is connectable to the carrier for immobilizing a body part of the patient on the carrier, the carrier and the immobilisation mask containing cooperating connection means, as described in the preamble of the first claim.

This invention also concerns an immobilisation mask for immobilizing a body part of a patient on a carrier, the immobilisation mask containing a sheet of a thermoplastic material for receiving the body part to be immobilized, and connection means for connecting the immobilisation mask to the carrier.

This invention further concerns a profile for connecting an immobilisation mask for immobilizing a body part of a patient on a carrier, to the carrier.

The use of masks or similar means for positioning one or more body parts of a patient in a predetermined position on the carrier and immobilizing of the body part in this position in a predetermined posture during the treatment of the patient with radiation, such as for example with Intensity Modulated Radiation Therapy (IMRT), Image Guided Radiation Therapy (IGRT), stereotactic radiation therapy or surgery and proton therapy, is well known. With such techniques, the accurate and reproductive positioning of the body part to be treated of a predetermined position in a predetermined posture is of utmost importance. As use, is often made of a radiation source that has the shape and/or size of the tissue to be treated, it is therefore essential that the radiation is directed at the tumour, and to spare the surrounding healthy tissue. Deviations of a few mm may result in that not the tumour is irradiated, but the surrounding healthy tissue, which is thus destroyed or damaged. This is undesirable.

Treatment and imaging techniques that make use of charged, heavy particles such as protons, offer the advantage in comparison to other radiation sources such as electrons, photons, gamma of X-radiation that the radiation dose can be better directed to the tissue to be treated. Proton beams usually scatter less in the tissue to be treated an exhibit a low lateral dispersion, which limits the risk of lateral damage. This offers the advantage that a directed killing of tumour cells can be obtained and that the surrounding healthy tissue can be spared. In order to enable the directed killing of unwanted tissue with both more or less dispersing radiation beams, an accurate positioning of the body part to be treated in relation to the radiation source is essential. To minimize the risk of unwanted irradiation of surrounding tissue, it is not only required that the body part is accurately positioned in the desired posture in relation to the radiation source during the treatment, but also that the body part is fixated during the treatment in the intended position in the intended posture and that the range of motion during the treatment is limited to a minimum. An accurate and reproducible positioning is of importance during fractionated treatment in which the patient is administered repeated radiation doses of less than optimal dose with in-between time intervals of typically a couple of days or weeks, to ensure sufficient radiation of the area to be treated and to limit the irradiation of healthy tissue to a minimum. An accurate positioning is also exceptionally important during a single treatment, in which a large radiation dose is administered once. Various tools have been designed to realize an accurate and reproducible positioning of a body part in relation to a radiation source, both with regard to the position as well as the posture, and thereby also to limit the range of motion of the body part to a minimum.

Traditionally, a patient is placed on a table, on which a base plate or carrier is located. Base plates that are used with radio therapy are often manufactured as a sandwich structure with a foam core and carbon fibre skin. Immobilizing a body part of a patient such as for example the head or any other body part, on a particular place on the table in a predetermined posture is realized by attaching an immobilisation mask to the base plate or the table. The immobilisation mask has a shape that closely matches the body part to be immobilized, to limit the range of motion of the body part to be immobilized.

EP1537831 in the name of Orfit discloses a non-invasive immobilisation mask for immobilizing for example the head of a patient in a predetermined position on a base plate or carrier, in a predetermined posture. The mask is manufactured out of a sheet of a thermoplastic material which is formed such that it matches the shape of the body part to be immobilized as closely as possible. To enable optimum design, a thermoplastic material is chosen with a low melting temperature, such that the material can be shaped directly onto the body part. An example of a suitable material is ε-polycaprolactone. Means are provided to at least a section of the edge of the mask for attaching the mask to the base plate. The attachment means disclosed in EP1537831 comprise a rail that extends along the edge or edges of the mask, the rail engages a corresponding slot in the base plate. The presence of the slot implies that the homogeneity of the material from which the base plate is made up, is interrupted, such that the homogeneity of the radiation beam that reaches the patient is adversely affected.

To meet the special requirements set by the radiation therapy concerning the immobilizing of the patient in relation to the radiation source and, especially with proton therapy, homogeneity of the radiation that reaches the tissue to be treated, a so-called base plate or carrier is designed that supports the patient well.

An example of a base plate or carrier, used in practice, is provided along the edge with a plurality of openings for receiving connection pins that fit into corresponding openings in the edge of the immobilization mask for attaching the mask onto the base plate. The pins are generally commercially available and know many applications in various fields of application. They are, for example, manufactured from polyoxymethylene or another plastic, they are relatively small and have the disadvantage that they exhibit a low mechanical strength and that they break often when establishing the connection. As a result, not only the number of times that the pins can be reused, is limited, but also there is the problem that the pins can loosen during the treatment, which would compromise the accurate positioning of the patient. In addition, the mask is subjected to considerable stress when it is connected to the carrier, which greatly hampers the application of the connection pins.

Consequently, there is a need for attachment means for attaching an immobilisation mask to a carrier or base plate for the support of the patient, that exhibits an improved mechanical strength.

The aim of this invention consists therefore in supplying an assembly of a carrier for supporting a patient and an immobilisation mask, connectable with the carrier, wherein means are provided for attaching the immobilisation mask to the carrier that exhibit an improved mechanical strength and a longer service life.

This is achieved according to the invention with an assembly that displays the mechanical characteristics of the body of the first claim.

To this end, the assembly of this invention is characterized in that connection means contain at least one profile that is provided on a first side to be connected to, or even for example connected to, the immobilisation mask, and on a second side opposite the first side contains first connection means for connecting the immobilisation mask to the carrier, wherein the first connection means are provided to engage, in the connected state, corresponding connection means along the outer edge of the carrier.

A profile that engages an outer edge of the carrier is simple to attach, also if a tensile force must be exerted onto the profile and/or the mask with which the profile is connected, to establish the connection. With a profile that engages the outer edge of the carrier, such a force is easier to exert then with the use of relatively small pins, which detach relatively easy when subjected to a large tensile force. To ensure that mainly the tissue to be treated is irradiated and the risk of undesired irradiation of surrounding tissue is kept to a minimum, it is of importance that the body part to be immobilized is fixated during the treatment at the intended place and in the intended posture and that the range of motion during the treatment is limited to a minimum. Therefore, it is of importance that the immobilisation mask closely fits the body part to be immobilized, which implies that the material of the mask, but also the profile, in the connected state with the carrier, are subjected to great tensile force and stress. The connection means or the profile of this invention enable, despite a high tensile force on the profile, to establish and maintain a connection.

The profile preferably engages in the thickness direction onto the carrier, this is usually the direction wherein the tensile force and tension onto the immobilisation mask and profile are the greatest. Since the profile engages in the thickness direction, optimal transfer of forces from the mask to the carrier is accomplished.

The profile preferably contains a first lower lip which is provided, in the connected state, to extend along a lower surface of the carrier, wherein the edge of the carrier is held between said upper and lower lip. In a further preferred embodiment, this lip is resiliently arranged to facilitate the establishment and severing of the connection.

To promote an accurate and reproducible positioning of the profile onto the carrier, on a lower surface of the carrier a guide is preferably provided for guiding the displacement of the lower lip to the position where the lip engages the carrier, at the connection of the immobilisation mask to the carrier.

The aim of this invention consists as well in providing an immobilisation mask that is provided with connection means for connecting the mask to the carrier, wherein the connection means exhibit an improved mechanical strength and longer operating life.

The immobilisation mask according to the invention and preferred embodiments thereof are disclosed in the attached claims, figures and figure description.

The aim of this invention consists further in connection means, in particular in providing a profile for attaching an immobilisation mask to a carrier for supporting a patient, wherein the connection means exhibit improved mechanical strength and a longer operating life.

The profile according to the invention and preferred embodiments thereof are disclosed in the attached claims, figures and figure description.

The invention is illustrated in the accompanying drawings which display preferred embodiments and describe the profile, the immobilisation mask provided with a profile, and an assembly of a carrier, an immobilisation mask and profile for the connection of the immobilisation mask to the carrier or base plate or carrier on which the patient rests. The invention is further illustrated in the description of the figures of these preferred embodiments. In the following figure description, the same reference numbers refer to the same elements.

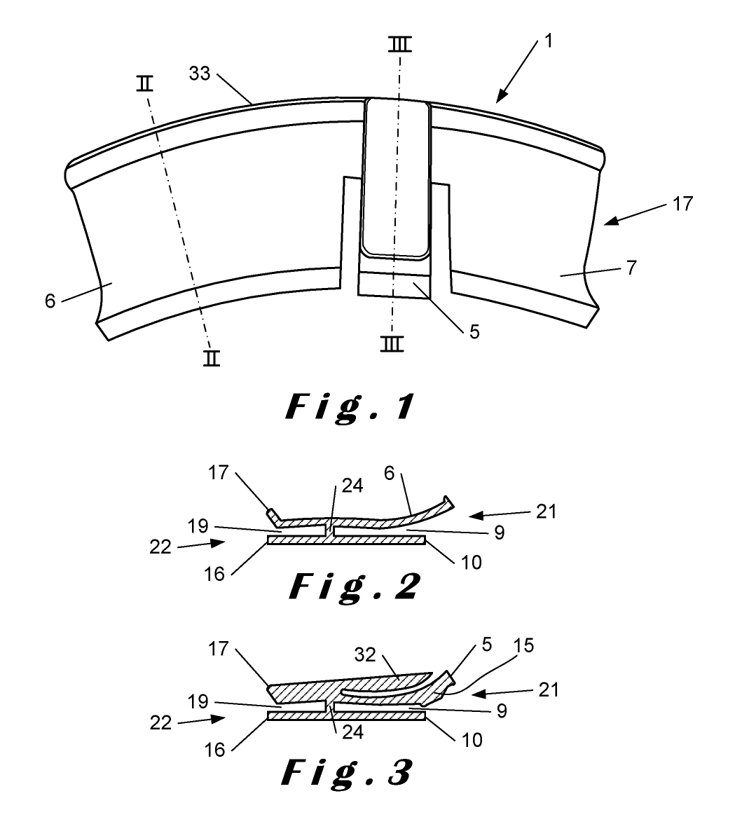

FIG. 1 shows a view of the lower side of the connection means (profile) for connecting an immobilisation mask with the carrier for supporting a patient or a portion thereof.

FIG. 2 shows a cross-section along the line II-II in FIG. 1.

FIG. 3 shows a cross-section along the line III-III in FIG. 1.

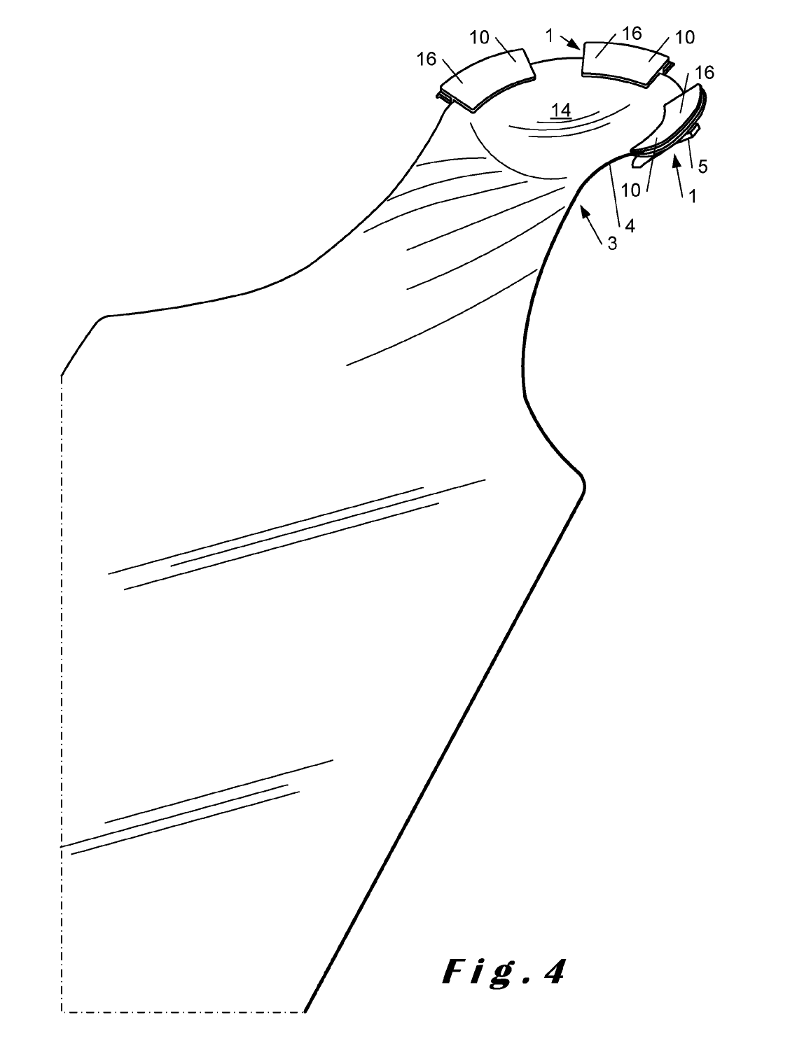

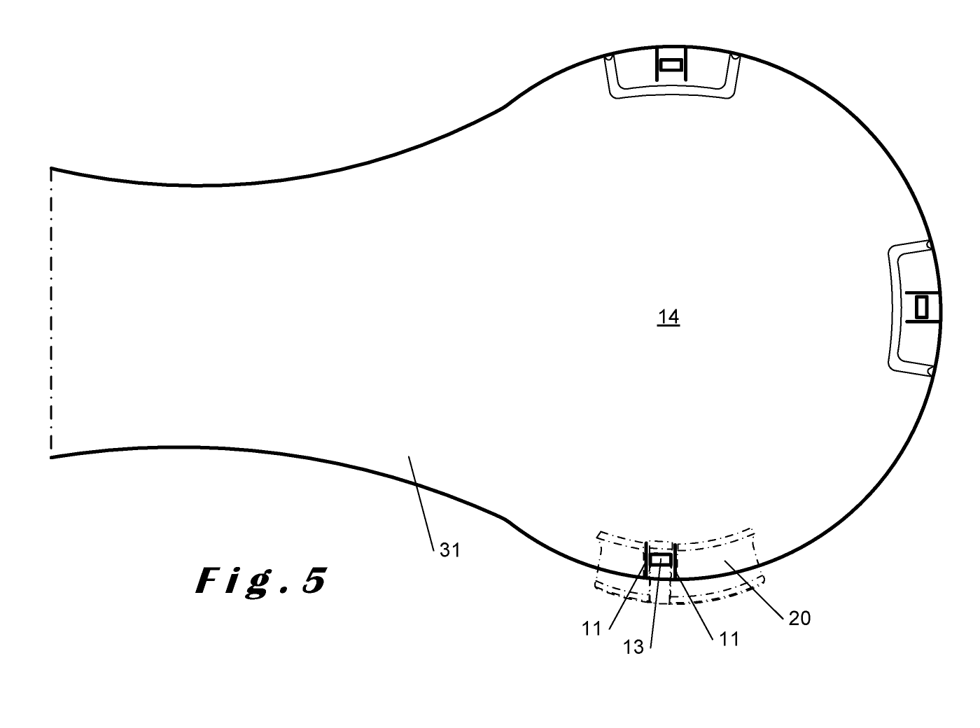

FIG. 4 shows a view to the upper side of a carrier.

FIG. 5 shows a view to the lower side of a carrier.

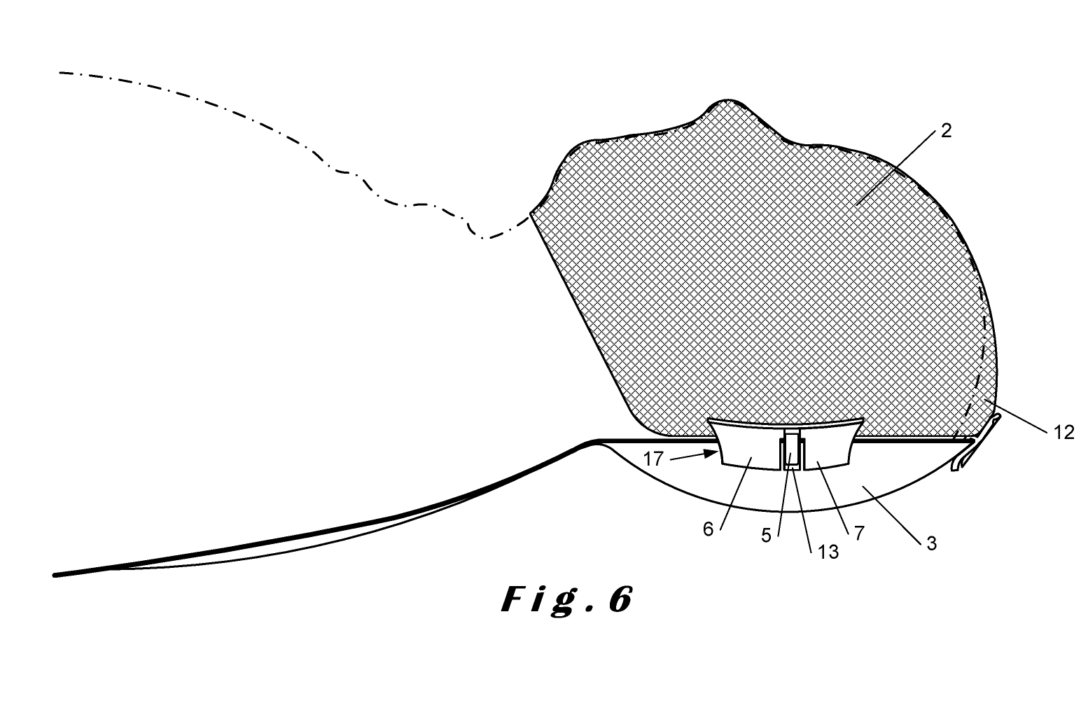

FIG. 6 shows an immobilisation mask in the connected state with the carrier.

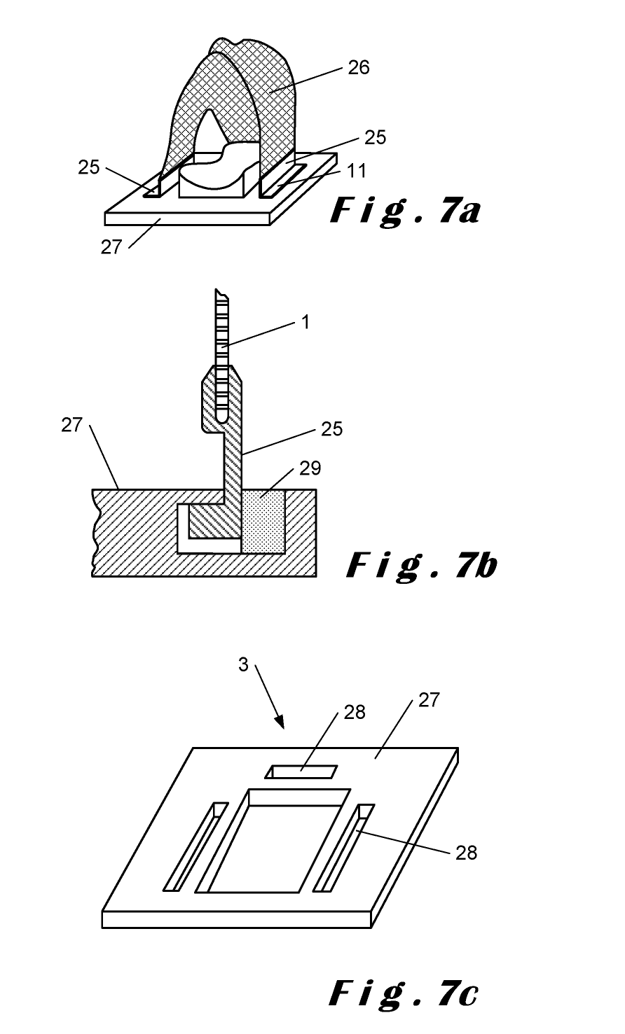

FIG. 7a shows a view to the immobilisation mask connected with a carrier according to the state of the art. FIG. 7b shows a cross-section of FIG. 7a. FIG. 7c shows a view of the carrier used in FIG. 7a.

FIGS. 7a, 7b and 7c show the way in which in the state of the art established a connection between a mask 26 for immobilizing a body part of a patient, for example the head, onto a carrier 27, for example a carrier on which the patient rests, is established. As is shown in FIG. 7a it is known from the state of the art to provide an L-shaped profile 25 along the edge of the mask that engages an opening or slot 28, provided in the surface of the carrier, in the region of the position where the patient rests. The slot 28 needs to be relatively wide, to enable the displacement of the L-shaped profile through the slit to enable its application. To fixate the position of the profile into the slot and to prevent displacement, it is necessary to apply a positioning block 29 or the like into the slot. The presence of the slot, the L-shaped profile and the positioning block disrupt the homogeneity of the material of the carrier at the position where the patient is located, so that there is a risk that the desired radiation dose into the tissue is not achieved.

A view to a upper side of a preferred embodiment of a carrier or base plate 3 for supporting a patient in radiation therapy is shown in FIG. 4. The preferred embodiment shown in FIG. 4 of the carrier 3 contains a top support surface 14 for supporting the patient. With “along” is meant that the connection means are located at a distance of the edge 4 of the carrier 3 or may be provided in one piece with the carrier, dependent on the nature of the connection means 21 on the profile. Along the edge of the carrier 3, preferably connection means 13 extend at least at one position, that are provided to cooperate with the connection means 1 for the connection of the immobilisation mask 2 with the carrier 3. Along the edge 4 of the carrier 3, there is provided preferably on at least one position, an opening 13 for receiving connection means 1 for attaching the immobilisation mask 2 to the carrier 3. More preferable, two or more openings 13 are provided. The amount of openings 13 is usually chosen such that a sufficient amount of profiles 1 can be applied to enable a stable and accurate fixation of the position and posture of the body part of the patient and to allow not more than the desired range of motion. The openings 13 may be applied at random positions along the edge 4 of the carrier. However, preferably, the openings 13 are applied symmetrically relative to the longitudinal axis of the carrier to attain optimal distribution of the tension and tensile force over the immobilisation mask 2 and to enable optimal derivation thereof to the carrier 3 (see FIG. 4). It is further possible to choose the amount of openings 3 such that the carrier 3 is suitable for immobilizing multiple body parts with varying size, Every other arrangement of openings 13 considered suitable by the person skilled in the art may be chosen.

Furthermore, the carrier 3 along the edge 4 is preferably, at least one position 20, deformed for receiving a profile 1 for attaching the immobilisation mask 2 to the carrier 3. The deformation 20 is preferably such that it is sufficiently large to receive the upper lip 10 of the profile 1 in such a way that the upper surface of the upper lip 10 is flush with the support surface 14 of the base plate or carrier 3 to increase the comfort of the patient. The presence of the deformation 20 hampers the displacement of the profile 1 along and relative to the edge of the carrier 4 and contributes in this way to the provision of a stable, accurate and reproducible connection to the immobilisation mask 2 with the carrier. The deformation also counteracts the possibility of rotation of the profile 1 relative to the carrier 3. Preferably, two or more deformations 20 are provided. The number of deformations 20 is usually chosen such that a sufficient amount of profiles can be applied to enable a stable and accurate fixation of the position and posture of the body part of the patient and to allow no more than the desired range of motion. The deformations 20 can be located at random positions along the edge of the carrier. However, preferably the deformations 20 are applied with some symmetry to realize optimal distribution of tension and tensile force over the immobilisation mask 2 and to enable optimal derivation thereof to the carrier 3 (see FIG. 4).

The dimensions and shape of the deformations 20 may be random. The deformation 20 preferably has the shape of a recess relative to the upper side of the carrier. In other words, the upper surface of the deformation 20 is preferably lowered relative to the upper surface 14 of the carrier. This is shown in FIG. 5. Preferably, the dimensions of the deformations 20 in the longitudinal direction along the edge 4 of the carrier and the dimensions of the deformation 20 in the traverse direction on the carrier 4 are aligned with the dimensions of the profile 1 or connection means that are provided to be incorporated into this deformation. The deformation 20 preferably has nearly the same dimensions as the profile 1 or is slightly larger than the profile 1, such that the profile 1 or at least the upper lip 10 of the profile can be incorporated in the deformation 20. The deformation 20 may function, at the application of the profile, as a guide for guiding the displacement of the profile 1 during the establishment of the connection between the mask 2 and the carrier 3. Since the raised edge of the deformation forms a barrier, the displacement of the profile 1, once installed, is opposed, and the deformation 20 contributes to the stable and accurate fixation of the position and posture of the body part of the patient so as to allow no more than the intended range of motion.

A view to the lower side of a preferred embodiment of a commonly used carrier 3 for supporting a patient, especially in proton therapy, is shown in FIG. 5. At a position between the edge 4 of the carrier and the opening 13, a guide 11 is preferably provided at the lower side of the carrier for guiding the displacement of the profile 1 at the establishment of the connection of the immobilisation mask 2 with the carrier 3, in particular for guiding the displacement of the first lower lip 5 on the profile 1. A preferred embodiment of the guide 11 has the shape of two opposite protrusions or rails, in between which the lip 5 of the profile 1 is displaceable at the establishment of the connection. Once the connection has been established, the protrusions counteract a displacement of the lip 5 in a direction along the edge and therefore contribute to a stable and accurate fixation of the position and posture of the body part of the patient. Instead of the protrusions or rails 11, an alternative guide, for example a slot or any other alternative deemed suitable by the person skilled in the art, may be used that is adapted to embodiment of the lower lip 5.

The connection means preferably comprise a profile 1 with first connection means 21 at a first side of the profile along which the profile is connectable to the carrier 3, and second connection means 22 at a second side of the profile 1 opposite the first side along which the profile 1 is connectable to the immobilisation mask 2. The first connection means 21 are provided, in the connected state, to engage an outer edge of the carrier. Such connection means enable to establish a connection between the immobilisation mask 2 and the carrier 3, also when the profile is subjected to high tension. The first connection means 21 preferably engage in the thickness direction the outer edge of the carrier. In a possible embodiment, the connection means 21 exercise a clamping force onto the carrier. In another possible embodiment, the first connection means 21 exercise a clamping force onto the carrier 3.

The first connection means 21 preferably comprise an upper lip 10 that is provided to extend, in the connected state, along an upper surface or side of the carrier 14. Preferably, the upper lip 10 is incorporated in a corresponding deformation 20 along the upper surface 14 of the carrier 3. To enable a greater transmission of forces between the immobilisation mask 2 and the carrier 3, the upper lip 10 preferably extends in the longitudinal direction along the edge 4 of the carrier 3. The upper lip 10 may, for example, be curved to optimally follow the shape of the immobilisation mask 2, the upper lip 10 may however also be flat and rectangular or may take any other appropriate shape. The edge 33 of the upper lip may, for example, be curved to optimally follow the shape of the edge of the immobilisation mask as shown in FIG. 1. The upper lip 10 increases the stability of the mask in the connected state with the carrier, since a tilting movement of the profile is counteracted.

The first connection means 21 preferably also comprise at least one lower lip 5 that is provided to extend in the connected state along a lower surface or lower side of the carrier 31. More preferably, the first connection means 21 comprise a lower lip 5 that is provided on the side directed to the carrier with a protrusion 15 that is provided, in the connected state, to engage corresponding connection means on the lower side of the carrier 3. Preferably, protrusion 15 engages a corresponding opening 13 in the carrier 3. The opening 13 may extend throughout the entire thickness of the carrier 3 or over a section thereof. The first connection means 21 further preferably comprise on a first side of the lower lip 5 a second lower lip 6 which preferably extends in the longitudinal direction along the edge of the carrier 3. The first connection means 21 further preferably comprise at a second side of the lower lip 5 opposite the first side in transverse direction of the lower lip 5, a third lower lip 7 which preferably extends in the longitudinal direction along the edge of the carrier 3. With a resiliently arranged lip 5, an easily detachable, connection is provided between the profile 1, in particular between the profile and the carrier 3. The second and third lip 6, 7 may have the same or different shapes, but preferably have a corresponding shape. The second and third lower lips 6, 7 may, for example, be curved to optimally follow the shape of the immobilisation mask, they may however also be rectangular or may take any other appropriate shape. The presence of the second and third lower lip 6, 7 increases the stability of the mask in the connected state with the carrier, since a tilting movement of the profile 1 is counteracted.

Preferably, the outer surface of lip 5 further contains a second lip 32 that counteracts or even prevents a movement of the lip 5 that is directed away from the space 9.

The opening 13 can be arranged centrally in the deformation 20, or asymmetrically.

The at least one upper lip 10 and at least one lower lip 5, 6, 7, of the first connection means 21 are preferably arranged at a distance from one another and are connected to each other at a connection position 24. Thereby, the first distance between the at least one upper lip 10 and at least one lower lip 5, 6, 7, is preferably chosen such that, at one first side of the connection position 2, it is sufficiently large for receiving a section of the carrier 3 that extends along the edge 4 thereof. The second connection means 22 preferably contain also an upper and a lower lip 16, 17 that are connected to each other at the connection position 24. The upper and lower lip 16, 17 are preferably arranged at a second distance from each other that is sufficiently large for the inclusion of the material along the edge of the immobilisation mask. The first distance can be equal to the second distance, or may be different from it. Connection position 24 connects the first 21 and second connection means 22.

The first connection means 21 for connecting the profile to the carrier, preferably extends in a first direction relative to the connection position 24, in particular in the direction of the carrier 3. The second connection means 22 that connect the profile 1 to the immobilisation mask preferably extend in opposite direction relative to the first connection means 21 and the connection position 24, in particular in the direction of the immobilisation mask.

The carrier 3 may be provided in every material deemed suitable by the person skilled in the art. The carrier is for example conducted as a plate of carbon fibres impregnated with epoxy resin. However, other materials may be used.

The profile 1 may be provided in one piece with the immobilisation mask 2 or as a separate component. Preferably, it is provided in one piece with the mask 2 in order to absorb higher tensile forces on the mask with minimal risk of releasing the profile 1. The profile 1 may be provided in another material than the mask, such that for both components the material may be chosen that is optimally adapted to the selected function it performs. This offers, for example, the possibility to choose the material for the mask such that it may directly be shaped to the patient, and to choose the material of the profile 1 such that it exhibits an optimal mechanical strength and minimal deformation to ensure an accurate and reproducible positioning.

Suitable materials for manufacturing the immobilisation mask are for example thermoplastic elastomers, thermoplastic polyurethane, thermoplastic polyisoprene, thermoplastic polyesters, thermoplastic polyolefins, polyvinyl chloride, polystyrene, or a blend of two or more of these materials. Preferred are these polymers that have a relatively low softening temperature wherein the material is deformable, such that these are exceptionally suitable for the manufacturing of immobilisation mask that can directly be formed onto the body part to be immobilized. The body part to be immobilized then serves as a mould for shaping the immobilisation mask. Examples of suitable thermoplastic polyolefins comprise polyethylene, polypropylene or ethylene-propylene co-polymers. Examples of suitable thermoplastic polyesters comprise polyethylene vinyl acetate, polyacrylates or polymethacrylate, polymeric fatty acid esters, in particular poly-ε-caprolactone, for example poly-ε-caprolactone such as available at Perstorp (UK) under the trade name Capa®. Preferred are the polymers of the group of thermoplastic polyurethane, isotactic polypropylene, copolymers of ethylene with 1-butene, a copolymer of ethylene with 1-octene, poly-ε-caprolactone, poly-ε-caprolactone containing thermoplastic polyurethane, as well as mixtures of two or more of these materials. Poly-ε-caprolactone is especially preferred because it has a low melting point and is deformable at temperatures that the human body can tolerate.

Suitable materials for manufacturing the profile are ABS, polyoxymethylene, polyamide or other equivalent materials.

This invention also concerns an immobilisation mask for immobilizing a body part of the patient in a previously determined posture of the carrier 3, wherein the immobilisation mask is provided with a profile 1 as described above, for connecting the mask 2 to the carrier 3 for supporting the patient. Preferably, the profile 1 is attached to the mask 2 along the edge of the mask 12, by incorporating the edge 12 of the mask in the second space 19 at the second connection means 22. Attachment can be done, for example, by gluing, by welding, by means of a mechanical connection or by any other connection deemed suitable by the person skilled in the art. Preferably, the connection is established by gluing or welding. Preferably, the profile 1 is as described above. To enable an optimal positioning and immobilizing along the edge of the mask, usually multiple profiles 1 will be applied.

This invention further concerns the profile 1 for connecting an immobilisation mask 2 to the carrier 3, wherein the mask 2 is provided for immobilizing a body part of a patient in a predetermined posture on the carrier. Preferably, the profile 1 is as described above.

Connecting an immobilisation mask 2 to the carrier 3 upon which a patient rests, for immobilizing for example the head of the patient occurs by covering the head of the patient with the immobilisation mask that was shaped to the head of the patient. When use is made of an immobilisation mask on which along the edge a plurality of profiles are attached, a connection is established by incorporating the edge 4 of the carrier 3 in the first space 9 of the profile 1 at the side of the first connection means 21, preferably at the height of a recess 20 in the carrier 3. When establishing the connection, the profile is usually placed transversal to the edge 4 of the carrier 3 in the direction of the middle of the carrier 3. The displacement of the profile 1 may be guided by the edges of the recess or deformation 20 at the upper side of the carrier 14 and by the guide 11 at the lower side 31 of the carrier that guides the displacement of the first lip 5 along the lower side 31 of the carrier 3 till the protrusion 15 engages the carrier at the first lip 5 in the corresponding opening 13.

### LIST OF REFERENCE NUMBERS

1. profile
2. immobilisation mask
3. carrier for patient
4. edge carrier for patient
5. first lower lip
6. second lower lip
7. third lower lip
8.
9. first space
10. upper lip
11. guide
12. edge mask
13. opening in edge carrier for patient
14. support surface carrier for patient
15. protrusion on first lower lip
16. upper lip second connection means
17. lower lip second connection means
18.
19. second space
20. deformation in carrier
21. first connection means
22. second connection means
23.
24. connection position
25. L- shaped profile
26. immobilisation mask
27. carrier
28. slot in carrier
29. EVA block—
30.
31. lower side carrier
32. second lip
33. edge upper lip

1-31. (canceled)

32. Assembly for radiation therapy, for example proton therapy, of a carrier for supporting a patient, an immobilisation mask connectable with the carrier for immobilizing a body part of the patient onto the carrier and connection means for connecting the carrier with the immobilisation mask, wherein the immobilisation mask is manufactured from thermoplastic material, wherein the connection means contain at least one profile that is provided on a first side to be connected to the immobilisation mask and on a second side opposite the first, contains connection means for connecting the immobilisation mask to the carrier, wherein the first connection means are provided, in the connected state, to engage corresponding connection means along the outer edge of the carrier and wherein the first connection means comprise a first lip that is provided, in the connected state, to extend along the carrier.

33. Assembly according to claim 32, wherein the at least one profile is connected to the immobilisation mask.

34. Assembly according to claim 32, wherein the immobilisation mask is manufactured from a thermoplastic material, chosen from the group, consisting of thermoplastic elastomers, thermoplastic polyurethane, thermoplastic polyisoprene, thermoplastic polyesters, thermoplastic polyolefins, polyvinyl chloride, polystyrene, or a blend of two or more of these materials.

35. Assembly according to claim 34, wherein the immobilisation mask is manufactured from thermoplastic polyesters.

36. Assembly according to claim 34, wherein the immobilisation mask is manufactured from suitable thermoplastic polyolefins, selected from the group consisting of polyethylene, polypropylene, or ethylene-propylene co-polymers, selected from the group of polyethylene vinyl acetate, polyacrylates or polymethacrylate, polymeric fatty acid esters, in particular poly-8-caprolactone, polymers found to be suitable from the group of thermoplastic polyurethane, isotactic polypropylene, copolymers of ethylene with 1-butene, a copolymer of ethylene with 1-octene, poly-G-caprolactone, poly-C-caprolactone containing thermoplastic polyurethane, as well as mixtures of two or more of these materials.

37. Assembly according to claim 32, wherein the immobilisation mask is manufactured from polymers that have a softening temperature at which the material is deformable, such that they are suitable for the manufacturing of immobilisation masks that can be directly shaped onto the body part to be immobilized, wherein the body part to be immobilized serves as mould for shaping the immobilisation mask.

38. Assembly according to claim 32, wherein the immobilisation mask is manufactured from poly-6-caprolactone.

39. Assembly according to claim 32, wherein the first connection means of the profile and the outer edge of the carrier contain complementary, co-operating connection means.

40. Assembly according to claim 32, wherein the first connection means comprise an upper lip that is provided, in the connected state, to extend along an upper surface of the carrier, and the lower lip is provided, in the connected state, to extend along a lower side of the carrier, wherein the edge of the carrier is held between said lower and upper lip.

41. Assembly according to claim 32, wherein the first connection means, in the connected state, engage the corresponding connection means along the outer edge of the carrier.

42. Assembly according to claim 40, wherein the first lower lip is arranged resiliency.

43. Assembly according to claim 40, wherein the first lower lip at a side directed to the carrier is provided with a protrusion that is provided, in the connected state, to engage the corresponding connection means along the outer edge of the carrier.

44. Assembly according to claim 43, wherein the corresponding connection means along the outer edge of the carrier comprise an opening for receiving the protrusion.

45. Assembly according to claim 40, wherein the first connection means further comprise a second and third lower lip at opposite sides of the first lower lip, wherein the second and third lower lip are provided to extend along the lower side of the carrier.

46. Assembly according to claim 40, wherein at the lower side of the carrier a guide is provided for guiding the displacement of the first lower lip to the position where the first lower lip of the carrier engages at the establishment of the connection.

47. Assembly according to claim 40, wherein the carrier is deformed along the edge for receiving the lower lip.

48. Assembly according to claim 40, wherein the lower lip is elongated in a direction which extends along the edge of the carrier.

49. Immobilisation mask for immobilizing a body part of a patient on a carrier, wherein the immobilisation mask contains a sheet of a thermoplastic material for receiving the body part to be immobilized, and is provided with connection means for connecting the immobilisation mask to the carrier, characterized in that the connection means contain at least one profile that at a first side contains first connection means for connecting the profile to the carrier, wherein the first connection means are provided, in the connected state, to engage corresponding connection means for connecting the immobilisation mask to the profile, wherein the first connection means are provided, in the connected state, to engage corresponding connection means along the outer edge of the carrier.

50. Immobilisation mask according to claim 49, wherein the first connection means on the profile contains connection means that are complementary to and are provided to cooperate with corresponding connection means along an outer edge of the carrier.

51. Immobilisation mask according to claim 49, wherein the first connection means comprise a first lip that is provided to extend along the carrier in the connected state.

52. Immobilisation mask according to claim 51, wherein the first connection means comprise a upper lip that is provided to extend in the connected state along an upper surface of the carrier, and the first lip is provided to extend in the connected state along a lower side of the carrier and to engage the corresponding connecting means along the outer edge of the carrier.

53. Immobilisation mask according to claim 51, wherein the first lip is arranged resiliency.

54. Immobilisation mask according to the claim 51, wherein the first lower lip is provided, at a side directed at the carrier, with a protrusion that is provided, in the connected state, to engage the corresponding connection means along the outer edge of the carrier.

55. Immobilisation mask according to claim 51, wherein the first connection means further comprise a second and third lower lip at opposite sides of the first lower lip, wherein the second and third lower lip are provided to extend along the lower side of the carrier.

56. Immobilisation mask according to claim 51, wherein the upper lip is elongated in a direction that extends along the edge of the carrier.

57. Profile for connecting a carrier to the immobilisation mask for immobilizing a body part of a patient on the carrier, characterized in that the profile contains at a first side first connection means for connecting the profile to the carrier, and at a second side opposite to the, first side, second connection means for connecting the profile to the immobilisation mask, wherein the first connection means are provided, in the connected state, to engage corresponding connection means along the outer edge of the carrier.

58. Profile according to claim 57, wherein the first connection means comprise an upper lip that is provided, in the connected state, to extend along the upper surface of the carrier, and a first lower lip that is provided, in the connected state to extend along the lower side of the carrier and to engage the corresponding connection means at a lower side of the carrier.

59. Profile according to claim 57, wherein the lower lip is arranged resiliency.

60. Profile according to claim 57, wherein the first lower lip on a side directed to the carrier is provided with a protrusion that is provided, in the connected state, to engage the corresponding connection means at a lower side of the carrier.

61. Profile according to claim 57, wherein the first connection means further comprise a second and third lower lip at opposite sides of the first lower lip, wherein the second and third lower lip are provided to extend along the lower side of the carrier.

62. Use of the assembly according to claim 32.

63. Use of the assembly according to claim 62, wherein the immobilisation mask contains a sheet of a thermoplastic material for receiving the body part to be immobilized, and is provided with connection means for connecting the immobilisation mask to the carrier, characterized in that the connection means contain at least one profile that at a first side contains first connection means for connecting the profile to the carrier, wherein the first connection means are provided, in the connected state, to engage corresponding connection means for connecting the immobilisation mask to the profile, wherein the first connection means are provided, in the connected state, to engage corresponding connection means along the outer edge of the carrier.

64. Use of the assembly according to claim 62, wherein the profile contains at a first side first connection means for connecting the profile to the carrier, and at a second side opposite to the, first side, second connection means for connecting the profile to the immobilisation mask, wherein the first connection means are provided, in the connected state, to engage corresponding connection means along the outer edge of the carrier.
